(19) United States
(12) Patent Application Publication
(10) Pub. No.: US 2012/0136330 A1
(43) Pub. Date: May 31, 2012

(54) Disposable Absorbent Article Donning Efficiency

(76) Inventors: Daniel Robert Schlinz, Greenville, WI (US); Kathy Geralyn Richardson, Combined Locks, WI (US); Julie Ann Paveletzke, Appleton, WI (US); Heidi Kay Wudi, Appleton, WI (US)

(21) Appl. No.: 12/956,258

(22) Filed: Nov. 30, 2010

(51) Int. Cl. A61F 13/58; G09B 19/00

(52) U.S. Cl. 604/385.03; 434/262

(57) ABSTRACT

A pant design supports a method for applying a pre-fastened refastenable absorbent article to a subject without removing the subject's clothing. The method includes opening the transverse sides of the article, and threading the article between the legs such that the article is positioned between the feet and the knees. The method also includes forming a wear configuration by pulling the front and back side panels of the each transverse side around each leg such that a front side panel meets a back side panel, and fastening the front side panel to the back side panel of each transverse side. The method also includes drawing the article up into a wear position on the subject.

### BACKGROUND

The present disclosure relates generally to absorbent articles intended for personal wear, and more particularly to disposable absorbent articles.

Many absorbent articles intended for personal wear, e.g., such as diapers, training pants, feminine hygiene products, adult incontinence products, bandages, medical garments and the like are designed to be sufficiently absorbent to pull moisture from liquid body exudates including urine, menses, blood, etc. away from the wearer to reduce skin irritation caused by prolonged wetness exposure.

Training pants and adult incontinence products can come pre-assembled in a wear configuration to more closely resemble conventional underpants. In particular, front and back waist regions of such training pants are typically attached at a seam either permanently or refastenably (such as by a primary fastening system) to define a wear configuration of the pants having a waist opening and leg openings.

For such articles where the attachment is refastenable, such as diapers and training pants, a caregiver can find the article difficult to apply to the subject without removing the subject's shoes and bottom garments. This is particularly applicable to active children of toilet training age, and of adults. The present disclosure is directed to a process for making a pant-like absorbent garment having refastenable fasteners, such as hook and loop fasteners, on the side panels for ease of removal and donning of the absorbent garment without complete removal of a wearer's clothing.

### SUMMARY

There is a need, therefore, for a donning system provided for an absorbent article such as training pants for improved efficiency in donning the absorbent article.

Disclosed herein is a pant design that supports a method for applying a pre-fastened refastenable absorbent article to a subject without removing the subject's clothing, the article having first and second transverse sides and the subject having legs, feet, ankles, and knees. The method includes opening the transverse sides of the article, each transverse side having front and back side panels, and threading article between the legs such that the article is positioned between the feet and the knees. The method also includes forming a wear configuration by pulling the front and back side panels of the first transverse side around one leg such that a front side panel meets a back side panel, fastening the front side panel to the back side panel of the first transverse side, pulling the front and back side panels of the second transverse side around the other leg such that a front side panel meets a back side panel, and fastening the front side panel to the back side panel of the second transverse side. The method also includes drawing the article up into a wear position on the subject.

In one aspect, an absorbent article supports a method for simplifying the application of a pre-fastened, refastenable absorbent article to a subject, the article having first and second transverse sides and the subject having legs, feet, ankles, and knees. The method includes manufacturing a plurality of refastenable absorbent articles and assembling the plurality of articles into a package. The method also includes providing information to a consumer to instruct the consumer to open the transverse sides of the article, each transverse side having front and back side panels, thread the article between the legs such that the article is positioned between the feet and the knees, form a wear configuration by pulling the front and back side panels of the first transverse side around one leg such that a front side panel meets a back side panel, fastening the front side panel to the back side panel of the first transverse side, pulling the front and back side panels of the second transverse side around the other leg such that a front side panel meets a back side panel, and fastening the front side panel to the back side panel of the second transverse side, and draw the article up into a wear position on the subject.

In another aspect, an absorbent article supports a method for simplifying the application of a pre-fastened, refastenable absorbent article to a subject, the article having front and back side panels and the subject having legs, feet, ankles, and knees. The method includes manufacturing a plurality of refastenable absorbent articles and assembling the plurality of articles into a package. The method also includes providing a first graphic image associated with the package illustrating threading an unfastened article between the legs such that the article is positioned between the feet and the knees, providing a second graphic image associated with the package illustrating forming a wear configuration by depicting pulling the front and back side panels around one leg such that the front side panel meets the back side panel, and providing a third graphic image associated with the package illustrating drawing the article up into a wear position on the subject, wherein at least one of the graphic images includes a depiction of the subject wearing shoes, and wherein at least one of the graphic images includes a depiction of the subject without bottom garments removed.

In still another aspect, an absorbent article package includes secondary information for simplifying the application of a pre-fastened, refastenable absorbent article to a subject, the article having front and back side panels and the subject having legs, feet, ankles, and knees. The package includes an outer surface and a plurality of refastenable absorbent articles disposed within the package. The package also includes a first graphic image associated with the package and illustrating threading an unfastened article between the legs such that the article is positioned between the feet and the knees; a second graphic image associated with the package and illustrating forming a wear configuration by depicting pulling the front and back side panels around one leg such that the front side panel meets the back side panel; and a third graphic image associated with the package illustrating drawing the article up into a wear position on the subject, wherein at least one of the graphic images includes a depiction of the subject wearing shoes, and wherein at least one of the graphic images includes a depiction of the subject without bottom garments removed.

Other features of the disclosure will be in part apparent and in part pointed out hereinafter. Other objects and advantages of the present disclosure will become more apparent to those skilled in the art in view of the following description and the accompanying drawings.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a side perspective view of one aspect of a personal wear article in the form of a pair of training pants;

FIG. 2 is a perspective view of a package of absorbent articles of a type similar to that shown in FIG. 1, the package including instructional graphic images;

FIG. 3 is a perspective view of a plurality of absorbent articles of a type similar to that shown in FIG. 1;

FIGS. 4a-4c are a series of instructional graphic images as can be disposed on the package of FIG. 2; and

FIGS. 5a-5c are an alternative series of instructional graphic images as can be disposed on the package of FIG. 2.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

It is to be understood by one of ordinary skill in the art that the present discussion is a description of exemplary aspects of the present disclosure only, and is not intended as limiting the broader aspects of the present disclosure.

The term “line of weakness” as used herein, refers to any region or area of weakened material, preferably having a length, but not necessarily a defined width. A “line of weakness” can include linear and non-linear patterns, such as curvilinear patterns of weakness, or other shapes, such as circles, rectangles, and so forth. A line of weakness includes a perforation or other series of cuts, a thinning or breakage or separation of material, or a strip of a different kind of material bridging between adjacent portions of material that is more easily torn or broken than the adjacent portions, and that allows the user or manufacturer to separate the adjacent portions along the line of weakness. A line of weakness can further include a single extended slit or cut.

It should be understood that the terms “personal care product” or “personal care article” as used herein refers to any article used to control bodily fluids, and includes “absorbent products” and “absorbent articles.” These refer to any article configured to absorb and retain bodily exudates, including urine, bowel movements, blood and menses, and includes such a product in a packaged and unpackaged configuration. As such, personal care products, as used herein, includes without limitation, diapers, child toilet training pants, adult incontinence garments, male incontinence products, tampons, vaginal suppositories, pantiliners, pads, sanitary napkins, tissues, wipes, etc. Examples of commercially available personal care products include, without limitation, Poise® feminine care products, including pantiliners and pads, and Kotex® feminine care products, including pads, tampons and liners, all available from Kimberly-Clark Corporation, Neenah, Wis.

As used herein, the term “shape retaining” is intended to mean that the shape of the flexible package with articles contained therein is essentially the same as the shape of the package while the articles are being removed and with all of the articles removed from the interior compartment of the package. That is the sides of the package do not tend to fall inward towards the interior compartment or outward away from the interior compartment and the top surface does not fall inward towards the interior compartment. It is noted that a small degree, i.e., less than about a 20% variation in width and/or length of the top surface, comparing the length or width of the top surface while the package is full to the length or width of the top surface with the articles removed, is considered to be shape retaining. For example, for a package having a width of 10 cm at the top surface with the articles contained therein, if the package has a width at the same point of 8-12 cm after the articles are removed, the package is considered to be shape retaining.

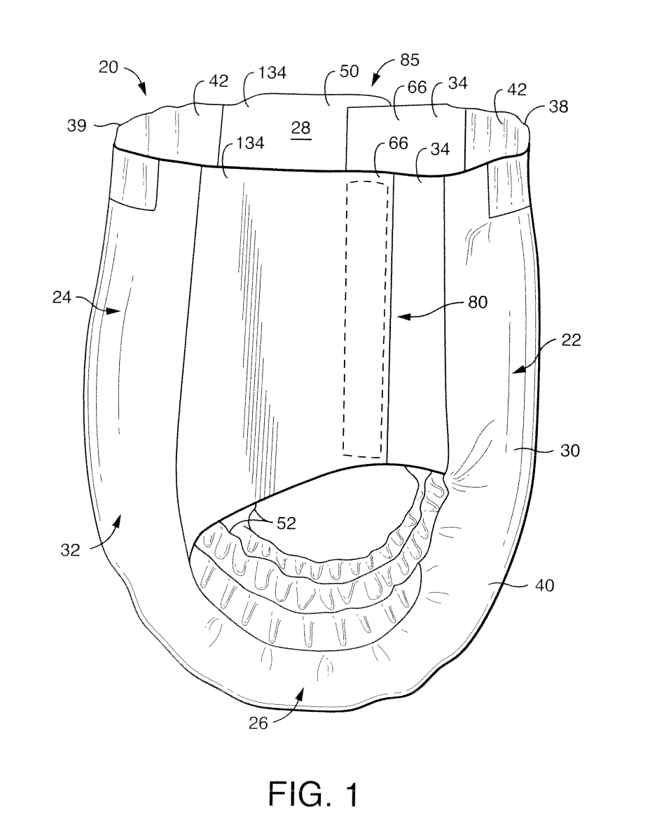

Referring now to the drawings and in particular to FIG. 1, a personal wear absorbent article according to one aspect is illustrated in the form of a pants-type article for wear about a wearer's waist, and more particularly in the form of children's toilet training pants, indicated in its entirety by the reference numeral 20. The term absorbent generally refers to articles that can be placed against or in proximity to the body of the wearer to absorb and/or retain various liquid wastes discharged from the body. The absorbent article can be disposable, which refers to articles that are intended to be discarded after a limited period of use instead of being laundered or otherwise restored for reuse. It is understood that the concepts described herein are suitable for use with various other pants-type articles such as adult incontinence articles, as well as other articles intended for personal wear such as clothing, diapers, feminine hygiene products, medical garments, surgical pads and bandages, other personal care or health care garments, and the like without departing from the scope of the present disclosure.

By way of illustration only, various materials and methods for constructing the training pants 20 are disclosed in PCT Patent Application WO 00/37009 published Jun. 29, 2000 by A. Fletcher et al; U.S. Pat. No. 4,940,464 issued Jul. 10, 1990 to Van Compel et al.; and U.S. Pat. No. 5,766,389 issued Jun. 16, 1998 to Brandon et al., which are incorporated herein by reference.

Training pants and incontinence garments can be difficult to put on a subject without removing the subject's shoes and bottom garments. This is particularly applicable to active children of toilet training age, and of adults. The present disclosure is directed to a process for making a pant-like absorbent garment having refastenable fasteners, such as hook and loop fasteners, on the side panels for ease of removal and donning of the absorbent garment without complete removal of a wearer's clothing.

For exemplary purposes, a pair of training pants 20 is illustrated in FIG. 1 in a fully pre-assembled (i.e., as assembled during initial manufacture) configuration (broadly referred to herein as a wear configuration of the pants, i.e., absorbent article). The training pants 20 includes a front waist region 22, a back waist region 24, a crotch region 26 extending longitudinally between and interconnecting the front and back waist regions along a longitudinal direction of the pants, an inner surface 28 configured for contiguous relationship with the wearer, and an outer surface 30 opposite the inner surface. The training pants 20 also has a pair of longitudinally opposite waist edges, respectively designated front waist edge 38 and back waist edge 39. The front waist region 22 is contiguous with the front waist edge 38, and the back waist region 24 is contiguous with the back waist edge 39.

The illustrated pants 20 includes a central absorbent assembly, generally indicated at 32. A pair of laterally opposite front side panels 34 extends outward from the absorbent assembly 32 at the front waist region 22 (thereby forming transversely outer portions of the front waist region, and more broadly in part forming transversely opposite sides of the training pants). Laterally opposite back side panels 134 extend outward from the absorbent assembly 32 at the back waist region 24 (thereby forming transversely outer portions of the back waist region, and together with the front side panels 34 further defining the sides of the pants).

The central absorbent assembly 32 of the illustrated aspect includes an outer cover 40 and a bodyside liner 42 connected to the outer cover 40 in a superposed relation by suitable means such as adhesives, ultrasonic bonds, thermal bonds or other conventional techniques. An absorbent structure is disposed between the outer cover 40 and the bodyside liner 42. A pair of containment flaps is secured to the bodyside liner 42 for inhibiting the lateral flow of body exudates. The central absorbent assembly 32 has opposite ends that form portions of the front and back waist edges 38 and 39.

The absorbent assembly 32 and side panels 34, 134 can include two or more separate elements, as shown in FIG. 1, or they can be integrally formed. Integrally formed side panels 34, 134 and absorbent assembly 32 would include at least some common materials, such as the bodyside liner 42, flap composite, outer cover 40, other materials and/or combinations thereof, and could define a one-piece elastic, stretchable, or nonstretchable pants 20. The pants 20 includes a longitudinal axis extending between the front and back waist edges 38 and 39 when the training pants 20 is in a laid-flat condition. The pants 20 also include a transverse or lateral axis that is perpendicular to the longitudinal axis and from one side to the other of the training pants 20.

With the training pants 20 in the fastened condition as illustrated fully in FIG. 1, the front and back side panels 34, 134 are attached to each other by a primary, or article fastening system 80 to define the pre-assembled three-dimensional wear configuration of the pants, having a waist opening 50 and a pair of leg openings 52. The front waist region 22 includes the portion of the training pants 20 that, when worn, is positioned at least in part on the front of the wearer while the back waist region 24 includes the portion of the training pants that is positioned at least in part on the back of the wearer. The crotch region 26 of the training pants 20 includes the portion of the training pants 20 that is positioned between the legs of the wearer and covers the lower torso of the wearer.

The front and back side panels 34 and 134 include the portions of the training pants 20 (and more particularly of the front and back waist regions 22, 24) that, when worn, are positioned on the hips of the wearer. The attached side panels 34, 134 thus broadly define the transversely opposite sides of the pants 20 at an engagement seam 66 along which the fastening system 80 releasably attaches the front and back side panels 34, 134. The waist edges 38 and 39 of the training pants 20 are configured to encircle the waist of the wearer and together define the waist opening 50. Portions of the side edges of the absorbent assembly 32 in the crotch region 26 generally define the leg openings 52.

The central absorbent assembly 32 is configured to contain and/or absorb exudates discharged from the wearer. For example, the containment flaps are configured to provide a barrier to the transverse flow of body exudates. Suitable constructions and arrangements for the containment flaps are generally well known to those skilled in the art and are described in U.S. Pat. No. 4,704,116 issued Nov. 3, 1987 to Enloe, which is incorporated herein by reference.

The outer cover 40 suitably includes a material that is substantially liquid impermeable. The liquid permeable bodyside liner 42 overlies the outer cover 40 and absorbent core, and can, but need not, have the same dimensions as the outer cover 40. The bodyside liner 42 is suitably compliant, soft feeling, and non-irritating to the child's skin.

As noted previously, the illustrated training pants 20 have front and back side panels 34 and 134 defining transversely opposite sides of the pants in the wear configuration of the pants. The side panels 34, 134 can be permanently attached along seams 66 to the central absorbent assembly 32 in the respective front and back waist regions 22 and 24. The side panels 34, 134 suitably, although not necessarily, include a stretchable material capable of stretching in a direction generally parallel to the transverse axis of the training pants 20. More suitably the side panels 34, 134 include an elastic material.

The absorbent structure can be any structure that is generally compressible, conformable, non-irritating to the wearer's skin and capable of absorbing and retaining liquid body exudates, and can be manufactured in a wide variety of sizes and shapes, and from a wide variety of absorbent materials commonly used in the art.

The article fastening system 80 includes laterally opposite first article fastening components adapted for refastenable engagement to corresponding second article fastening components. In one aspect, a front or outer surface of each of the article fastening components includes a plurality of engaging elements. The engaging elements of the first article fastening components are adapted to repeatedly engage and disengage corresponding engaging elements of the second article fastening components to releasably secure the pants 20 in its three-dimensional configuration. The article fastening system 80 provides an attachment of a strength sufficient to maintain the article in a wear configuration during use of the pants 20 by the wearer.

The article fastening components can include any refastenable fasteners suitable for absorbent articles, such as adhesive fasteners, cohesive fasteners, mechanical fasteners, or the like. In particular aspects, the article fastening components include mechanical fastening components for improved performance. Suitable mechanical fastening components can be provided by interlocking geometric shaped materials, such as hooks, loops, bulbs, mushrooms, arrowheads, balls on stems, male and female mating components, buckles, snaps, or the like.

In use, the training pants 20 are constructed and pre-assembled in their wear configuration, with the article fastening system 80 releasably attaching the front and back waist regions 22, 24 (and more particularly the front and back side panels 34, 134 in the illustrated aspect).

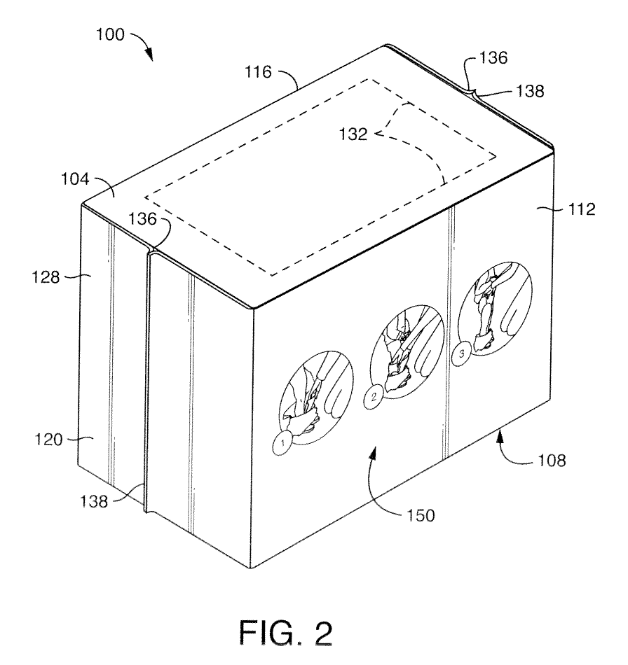

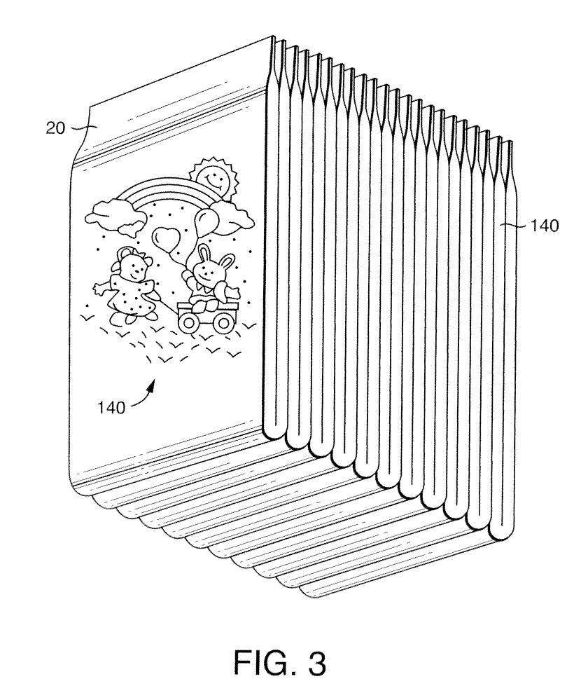

A package 100 of prefastened absorbent articles 20 is illustrated in FIG. 2. FIG. 3 illustrates a plurality 140 of absorbent articles 20 that are prefastened and prepared for insertion into the package 100. The package 100 includes a plurality 140 of prefastened absorbent articles 20 disposed within a sealed bag. The package 100 defines an interior space to receive the absorbent articles 20. The package 100 can but need not include a window for viewing the absorbent articles 20, and in particular the outer cover graphics 144 of the absorbent articles 20.

As illustrated in FIG. 2, the flexible package 100 has a top surface 104, a bottom surface 108, a front wall 112, a back wall 116, and a first side wall 120 and a second side wall 124. The first side wall 120 and the second side wall 124 are opposed to each other; as are the front wall 112 and the back wall 116, and the top surface 104 and the bottom surface 108. As shown, the front wall 112, the back wall 116, the two side walls 120, 124, the top surface 104, and the bottom surface 108 are directly or indirectly connected together to form the package 100, which has an interior compartment. The interior compartment is capable of containing a plurality 140 of articles 20. As shown in FIG. 3, the flexible package 100 has four side walls. It is not outside the present disclosure that the flexible package 100 has a single side wall that is continuous, two side walls, three side walls or more than four side walls. It is noted that in a common configuration, the flexible package 100 has four side walls and for purposes of understanding the present disclosure only, the flexible package 100 will be described in terms of a flexible package 100 having four side walls.

As shown, the package 100 includes an exterior surface 128. In this aspect, the package 100 includes one or more lines of weakness 132 that are designed to be broken open by the consumer. For instance, in one aspect, the package 100 can include vertical line(s) of weakness on the exterior surface 128 of the package 100. Alternatively, or in addition to the vertical line(s) of weakness, the package 100 can include horizontal line(s) of weakness also on the exterior surface 128 of the package 100.

The length of the line(s) of weakness 132 can also vary depending upon the particular application and the location of the line(s) of weakness 132. For instance, in one aspect, the line(s) of weakness 132 can extend along only a portion of the so length of the package 100 or of the width of the package 100. In one particular aspect, the exterior surface 128 of the package 100 can include a first vertical line of weakness offset from a second line of weakness.

The line(s) of weakness 132 can be formed by any means that will facilitate opening the package 100. Examples of a line of weakness 132 include, without limitation, a perforated line, a line formed by a plurality of openings, such as slots separated by a plurality of land areas, a line of reduced material thickness, a weakened line formed by joining two sections of material together, or any other suitable structural configuration. Each of these types of a line of weakness 132 can be formed by known methods including, for example, mechanically treating the package material, such as forming perforations in the package material.

The flexible package 100 can be constructed with at least one gusset 136, and desirably, with a pair of gussets 136. A gusset 136 is a member, for example a triangular member, capable of strengthening and/or enlarging the flexible package 100. The gusset 136 can be a separate piece of material or can be an extension of or integrally formed from the material from which the flexible package 100 is constructed. The gusset 136 can be viewed as a pocket, receptacle, cavity, or opening. The gusset(s) 136 can be located in the top of the package 100 and are exposed to make them visible to the ultimate consumer. Two gussets 136 when present can be located on the opposite sides of the top wall 104, on opposite sides of the bottom wall 108, or one in the top wall 104 and one in the bottom wall 108 to provide a natural location where the consumer can easily grasp the package 100. The gussets 136 can be formed in at least a portion of a side wall. Each gusset 136 is shown as having a triangular configuration, although variations of the triangular shape can be employed.

The flexible package 100 also includes a pair of seals 138, each formed in the opposing side walls 120, 124. The pair of seals 138 can be formed by a heat and pressure bond, by a thermal bond, by an ultrasonic bond, by adhesive, or by another suitable means. The purpose of the pair of seals 138 is to secure the pair of side walls 120,124 together whereby the front wall 112, the back wall 116, the pair of side walls 120, 124, and the top wall 104 create the internal compartment. The package 100 is designed to have the plurality 140 of articles 20 inserted into it via an open wall. After the articles 20 are positioned within the package 100, the open wall will then be sealed.

The package 100 can be prepared from a single piece of material or can be prepared from multiple pieces of material. If multiple pieces of material are used, the individual pieces must be joined using a suitable means. For example, the individual pieces can be joined by various conventional techniques, such as adhesive bonding, thermal bonding, ultrasonic bonding, welding, and the like. In another aspect, the panels are connected with mechanical fastening systems, such as sewing, stapling, riveting, and the like. In one aspect, the package 100 is formed from a continuous roll of material having a pre-formed gusset 136 and perforations.

The package 100 can be made from any one of a wide variety of suitable materials that are known to be sufficiently flexible to accommodate the desired number of articles 20 and have sufficient strength to hold and contain the articles 20 without breaking and without excessive bulging or stretching of the material. Materials include, but are not limited to, polymeric plastic films, foils, paper, paper composites, knitted or woven fabrics, nonwoven fabrics, and the like, or a combination thereof. Suitable materials can be made from polymeric materials such as polyethylene, polypropylene, polyester, nylon, and the like, as well as any combination thereof. In one aspect, the material is a low density polyethylene (LDPE) film. In another aspect, the material is an LDPE/LLDPE (linear low density polyethylene) film laminate. In yet another aspect, the material is an LDPE/MDPE (medium density polyethylene) film laminate, an LDPE/HDPE (high density polyethylene) film laminate, or the like. In still another aspect, a polyethylene/polypropylene combination is used. In one specific aspect, the material is a polyethylene film or film laminate having a thickness of between about 1 and about 5 mils (about 0.025 to 0.125 mm).

The present disclosure is also directed to a process for increasing the donning efficiency of a pant-like absorbent garment 20 having refastenable fasteners 80, such as hook and loop fasteners, on the side panels 34, 134 for ease of removal and donning of the absorbent garment 20 without complete removal of a wearer's clothing, particularly among seated, standing, or active subjects, such as active children of toilet training age and adults.

Existing methods for donning an absorbent article 20 are demonstrated in package instructions showing the article 20 being applied around the thighs of an adult. In addition, parents apply training pants 20 to a child by either removing the child's shoes and bottom garment and drawing up the article 20 similarly to regular underwear, or by applying the training pants 20 as they had previously applied a diaper—by opening the side seams 66 of the training pants 20, sliding the training pants 20 under their supine child, and then refastening the training pants 20. These methods require the parent or caregiver to either remove the shoes and bottom garment from a subject, or to stretch the side panels 34, 134 over the thighs of the subject in an attempt to refasten the article 20, resulting in distortion of the hook material that makes it difficult to quickly complete the refastening without having to repeatedly readjust the fastening.

Extensive testing has demonstrated that a more efficient donning process is to apply the article 20, including refastening the side seam 66, around the lower portion of the subject's leg, such as between the foot and the knee, where only minimal stretch is required of the side panels 34, 134 for refastening.

More specifically, the process relates to applying a pre-fastened refastenable absorbent article 20 to a subject without removing the subject's clothing. The absorbent article 20 includes first and second transverse sides 85, 90 (see FIG. 1) where front and back side panels 34, 134 meet to form a side seam 66. The caregiver first opens the transverse sides 85, 90 of the article 20 such that the article 20 is no longer in a wear configuration, and flattens the article 20 somewhat from its wear configuration. The caregiver then threads the article 20 between the subject's legs such that the article 20 is positioned between the feet and the knees, and more specifically adjacent the subject's ankles. The article 20 is positioned such that the front waist region 22 is generally in front of the subject's legs, the back waist region 24 is generally behind the subject's legs, and the crotch region 26 is generally between the subject's legs.

The caregiver then re-forms the wear configuration of the article 20 by pulling the front and back side panels 34, 134 of the first transverse side 85 around one leg such that a front side panel 34 meets a back side panel 134, and by fastening the front side panel 34 to the back side panel 134 of the first transverse side 85 to re-form a side seam 66. The caregiver also pulls the front and back side panels 34, 134 of the second transverse side 90 around the other leg such that a front side panel 34 meets a back side panel 134, and fastens the front side panel 34 to the back side panel 134 of the second transverse side 90 to re-form a side seam 66. Finally, the caregiver draws the article 20 up into a wear position on the subject.

Under current practice, a caregiver removes a currently-worn article from the subject prior to applying a new article. The subject is exposed to the public, elements, etc. while being cleaned and without an article 20. In another aspect of the method described herein, the new article can be applied to the subject around the lower portion of the subject's leg, such as between the foot and the knee, prior to removing the currently-worn article. In this aspect, a new article is ready to be drawn up as soon as the currently-worn article is removed, thus minimizing the exposure of the subject.

The difficulty in promoting the widespread use of the methods described in the present disclosure relates in large part to the difficulty in teaching the method to a caregiver. Because packages 100 of diapers, training pants, and incontinence garments 20 do not typically include instructions, caregivers apply such products in a way that is familiar to them: opening the article 20, sliding it under the subject, and fastening it about the subject's waist. Virtually every caregiver has changed a diaper or at least seen someone else change a diaper; that method is therefore the method caregivers know and use.

Retraining a population or even a generation of caregivers requires more than written instructions that will likely go unnoticed or will be lost in the shuffle of absorbent articles and caregiving. Testing demonstrates that prominently-displayed secondary information such as graphical images illustrating the method can be more effective than written instructions in conveying the method to a potentially-harried caregiver.

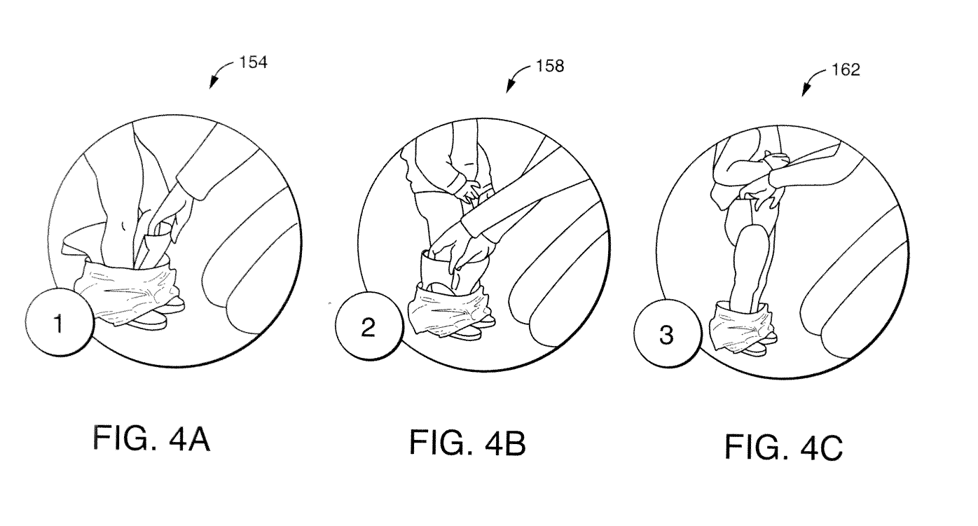

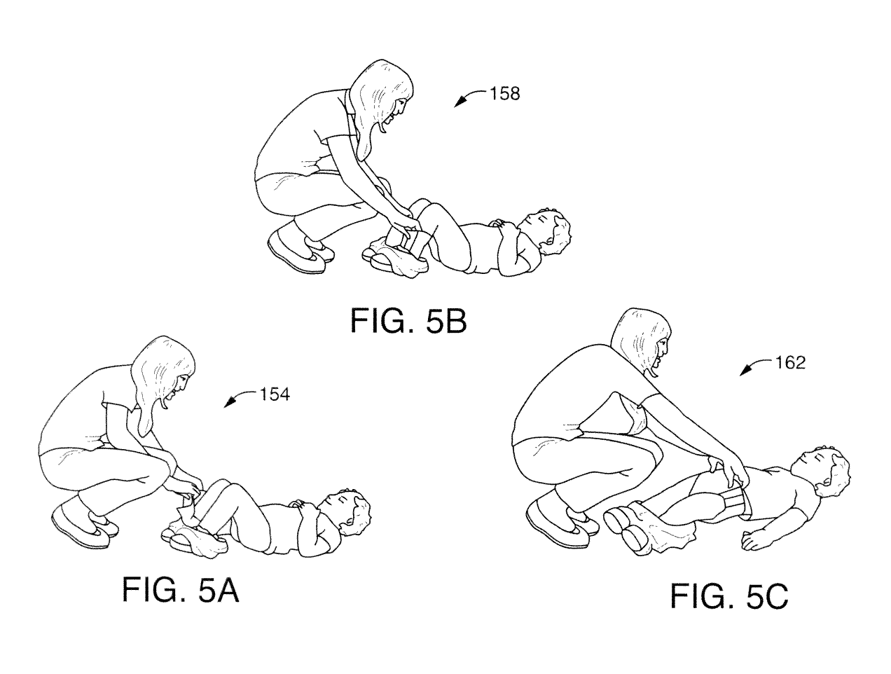

For effective retraining, instructions illustrating the method of the present disclosure can be presented in the form of one or more graphic images 150 (see FIG. 2). The graphic images 150 can be presented to the caregiver on the package 100, on an article 20, or on an insert disposed within or on the package 100. In one aspect of the present disclosure, the method is illustrated in a series of graphic images 150 such as those of FIGS. 4a-5c, each highlighting a particular step in the method.

In one example shown in FIGS. 4a-5c, a first graphic image 154 can illustrate threading an unfastened absorbent article 20 between the subject's legs such that the article 20 is positioned between the feet and the knees (see FIGS. 4a and 5a). A second graphic image 158 can illustrate forming a wear configuration by pulling the front and back side panels 34, 134 of at least one of the transverse sides 85, 90 around one leg such that the front side panel 34 meets the back side panel 134, and fastening the front side panel 34 to the back side panel 134 to re-form a side seam 66 (see FIGS. 4b and 5b). A third graphic image 162 can illustrate drawing the article 20 up into a wear position on the subject (see FIGS. 4c and 5c). Other graphic images can include only a portion of each of these steps, or a different combination of steps, or steps not included in these three graphic images 154, 158, 162.

Specific features of the method can also be illustrated in the graphic images 150 described above or in other graphic images. For example, as shown in FIGS. 4a, 4b, 5a, and 5b, at least one of the graphic images 150 can include a depiction of the subject wearing at least one shoe to demonstrate that the method can be performed without removing the subject's shoes. In another example shown in FIGS. 4a, 4b, 5a, and 5b, at least one of the graphic images 150 can include a depiction of the subject without bottom garments removed, such as with trousers pulled down to the subject's ankles but not removed. This depiction demonstrates that the method can be performed without removing the subject's bottom garment(s).

When introducing elements of the present disclosure or the preferred aspect(s) thereof, the articles “a,” “an,” and “the” are intended to mean that there are one or more of the elements. The terms “comprising,” “including,” and “having” are intended to be inclusive and mean that there can be additional elements other than the listed elements.

The disclosure has been described with reference to various specific and illustrative aspects and techniques. However, it should be understood that many variations and modifications can be made while remaining within the spirit and scope of the disclosure. Many alternatives, modifications and variations will be apparent to those skilled in the art in light of the foregoing description. Accordingly, this disclosure is intended to embrace all such alternatives, modifications, and variations that fall within the spirit and scope of the appended claims.

We claim:

1. A method for applying a pre-fastened refastenable absorbent article to a subject without removing the subject's clothing, the article having first and second transverse sides and the subject having legs, feet, ankles, and knees, the method comprising:
opening the transverse sides of the article, each transverse side having front and back side panels;
threading article between the legs such that the article is positioned between the feet and the knees;
forming a wear configuration by
pulling the front and back side panels of the first transverse side around one leg such that a front side panel meets a back side panel;
fastening the front side panel to the back side panel of the first transverse side;
pulling the front and back side panels of the second transverse side around the other leg such that a front side panel meets a back side panel; and
fastening the front side panel to the back side panel of the second transverse side; and
drawing the article up into a wear position on the subject.

2. The method of claim 1, wherein threading further comprises threading the article between the legs such that the article is positioned adjacent the ankles.

3. The method of claim 1, further comprising removing a previously-worn refastenable absorbent article from the subject, wherein the threading and forming acts are performed prior to the removing act.

4. A method for simplifying the application of a pre-fastened, refastenable absorbent article to a subject, the article having first and second transverse sides and the subject having legs, feet, ankles, and knees, the method comprising:
manufacturing a plurality of refastenable absorbent articles;
assembling the plurality of articles into a package; and
providing information to a consumer to instruct the consumer to
open the transverse sides of the article, each transverse side having front and back side panels;
thread the article between the legs such that the article is positioned between the feet and the knees;
form a wear configuration by pulling the front and back side panels of the first transverse side around one leg such that a front side panel meets a back side panel, fastening the front side panel to the back side panel of the first transverse side, pulling the front and back side panels of the second transverse side around the other leg such that a front side panel meets a back side panel, and fastening the front side panel to the back side panel of the second transverse side; and
draw the article up into a wear position on the subject.

5. The method of claim 4, wherein the instructing is conducted using graphic images.

6. The method of claim 5, further comprising disposing the graphic images on the package.

7. The method of claim 5, further comprising disposing the graphic images on a package insert, and further comprising coupling the package insert to the package.

8. The method of claim 5, further comprising providing a graphic image illustrating threading article between the legs such that the article is positioned between the feet and the knees.

9. The method of claim 5, further comprising providing a graphic image illustrating forming a wear configuration.

10. The method of claim 5, further comprising providing a graphic image illustrating drawing the article up into a wear position on the subject.

11. The method of claim 5, wherein at least one of the graphic images includes a depiction of the subject wearing a shoe.

12. The method of claim 5, wherein at least one of the graphic images includes a depiction of the subject without bottom garments removed.

13. The method of claim 4, wherein threading further comprises threading the article between the legs such that the article is positioned adjacent the ankles.

14. The method of claim 4, wherein providing information includes instructions to remove a previously-worn refastenable absorbent article from the subject, and wherein the consumer is instructed to thread and form prior to removing.

15. A method for simplifying the application of a pre-fastened, refastenable absorbent article to a subject, the article having front and back side panels and the subject having legs, feet, ankles, and knees, the method comprising:
manufacturing a plurality of refastenable absorbent articles;
assembling the plurality of articles into a package;
providing a first graphic image associated with the package illustrating threading an unfastened article between the legs such that the article is positioned between the feet and the knees;
providing a second graphic image associated with the package illustrating forming a wear configuration by depicting pulling the front and back side panels around one leg such that the front side panel meets the back side panel; and
providing a third graphic image associated with the package illustrating drawing the article up into a wear position on the subject, wherein at least one of the graphic images includes a depiction of the subject wearing shoes, and wherein at least one of the graphic images includes a depiction of the subject without bottom garments removed.

16. The method of claim 15, wherein the first graphic image further comprises illustrating threading the article between the legs such that the article is positioned adjacent the ankles.

17. The method of claim 15, wherein the first, second, and third graphic images are disposed on the outer surface of the package.

18. The method of claim 15, wherein the first, second, and third graphic images are disposed on an insert coupled to the package.

19. A package including secondary information for simplifying the application of a pre-fastened, refastenable absorbent article to a subject, the article having front and back side panels and the subject having legs, feet, ankles, and knees, the package comprising:
an outer surface;
a plurality of refastenable absorbent articles disposed within the package;
a first graphic image associated with the package and illustrating threading an unfastened article between the legs such that the article is positioned between the feet and the knees;
a second graphic image associated with the package and illustrating forming a wear configuration by depicting pulling the front and back side panels around one leg such that the front side panel meets the back side panel; and
a third graphic image associated with the package illustrating drawing the article up into a wear position on the subject, wherein at least one of the graphic images includes a depiction of the subject wearing shoes, and wherein at least one of the graphic images includes a depiction of the subject without bottom garments removed.

20. The package of claim 19, wherein the first graphic image further comprises illustrating threading the article between the legs such that the article is positioned adjacent the ankles.

21. The package of claim 19, wherein the first, second, and third graphic images are disposed on the outer surface of the package.

22. The package of claim 19, wherein the first, second, and third graphic images are disposed on an insert coupled to the package.
